(19) United States
(12) Patent Application Publication
(10) Pub. No.: US 2023/0149475 A1
(43) Pub. Date: May 18, 2023

(54) COMPOSITION FOR PREVENTING, IMPROVING, OR TREATING SARCOPENIA, COMPRISING TENEBRIO MOLITOR LARVAL PROTEIN OR HYDROLYSATE THEREOF AS ACTIVE INGREDIENT

(71) Applicant: Korea University Research and Business Foundation, Seoul (KR)

(72) Inventors: Yoo Kyung KIM, Seoul (KR); Eun Young OH, Seoul (KR); Young Kyun YOON, Seoul (KR)

(73) Assignee: Korea University Research and Business Foundation, Seoul (KR)

(21) Appl. No.: 17/916,198

(22) PCT Filed: Mar. 25, 2021
(86) PCT No.: PCT/KR2021/003739

§ 371 Date: Sep. 30, 2022

(30) Foreign Application Priority Data
Mar. 30, 2020 (KR) 10-2020-0038625
Mar. 24, 2021 (KR) 10-2021-0038130

(51) Int. Cl. A61K 35/64; A61P 21/00

(52) U.S. Cl. CPC A61K 35/64; A61P 21/00

(57) ABSTRACT

The present invention relates to: a pharmaceutical composition for preventing, improving, or treating muscle diseases, comprising a Tenebrio molitor larval protein or a hydrolysate thereof as an active ingredient; a food composition; a functional health food; and a feed composition. The Tenebrio molitor larval protein or hydrolysate thereof of the present invention can effectively inhibit myostatin expression, and thus, a composition comprising same as an active ingredient may be usefully used in the pharmaceutical, food, and feed industries as a composition for preventing, improving, or treating various muscle diseases caused by decreased muscle function, muscle loss, muscle atrophy, muscle wasting, or muscle degeneration.

### TECHNICAL FIELD

Disclosed are a pharmaceutical composition, a food composition, a health functional food, and a feed composition, each containing a mealworm protein isolate or a hydrolysate thereof as an active ingredient for prevention, alleviation, or treatment of a muscular disease.

### BACKGROUND ART

As of 2000, the Republic of Korea entered into an aging society as the elderly population accounted for 7.2% of its total population and is predicted to enter a super-aging society (more than 20% of the elderly population) in 2050 (Aged Statistics 2013, Statistics Korea). Human muscle mass decreases with aging (by 10-15% at 50-70 of age and by 30% or higher at 70-80 of age), with the concomitant degradation of muscular strength and muscular functions. The condition of age-associated muscular decline is referred to as geriatric sarcopenia. Geriatric sarcopenia is a major cause that limits the independent life of the elderly by causing activity and gait disturbances. In addition, sarcopenia lowers the basal metabolic rate, increases insulin resistance, promotes the onset of type 2 diabetes, and increases the risk of hypertension and cardiovascular disease by 3-5 times.

As the term “sarcopenia” was coined by Irwin Rosenberg in 1989, the concept of sarcopenia started. The term sarcopenia comes from the Greek word, “sari” (meaning muscle) and “penia” (meaning loss). Sarcopenia refers to the process of age-related decline of muscular strength with the decrease of muscle mass. In this regard, the term “muscle” means skeletal muscle and is irrelevant to smooth muscle. That is, sarcopenia means loss of skeletal muscle mass distributed mainly in the limbs and is distinct from cachexia, which is a state of significant muscle loss in the late stages of malignant tumors, muscle wasting due to acute diseases such as influenza, etc., or primary muscle disease. It should be considered resulting from a gradual decrease in skeletal muscle associated with aging.

Currently, there are no drugs approved for the treatment of sarcopenia, and drug repositioning technology for applying myostatin inhibitors or FDA-approved therapeutics for other diseases to sarcopenia is being developed.

Myostatin is a polypeptide functioning as a growth factor that is a member of the TGF-β superfamily. TGF-β includes many isoforms that are known to be involved in cell proliferation, apoptosis, differentiation, and bone formation and maintenance (Massague & Chen, 2000). Myostatin, also known as growth differentiation factor (GDF) 8, is involved in the growth and development of tissues, acting by activating the Smad signaling pathway. In addition, the growth factor is reported to affect osteogenesis and osteoanagenesis by upregulating p21 gene, which causes the inhibition of the cell cycle and myoblast proliferation. Myostatin is produced mainly in skeletal muscle cells and causes muscle loss and muscular strength reduction in a self-secretion manner. Myostatin also downregulates the expression of IGF-1 or follistatin, which are implicated in muscular hypertrophy, thereby inhibiting the protein synthesis and proliferation of myoblasts.

Conventionally, antibodies for inhibiting only the function of myostatin have been prepared for the treatment of sarcopenia, but side effects have been reported, and active studies on anti-myostatin materials using natural products are ongoing.

Under this background, intensive and thorough research, conducted by the present inventors with the aim of developing an agent capable of effectively treating sarcopenia, resulted in the finding that a mealworm protein isolate and a hydrolysate thereof can prevent or treat sarcopenia by efficiently inhibiting the expression of myostatin, leading to the present disclosure.

### DISCLOSURE OF INVENTION

### Technical Problem

Therefore, an aspect of the present disclosure is to provide a pharmaceutical composition capable of effectively preventing or treating a muscular disease.

Another aspect of the present disclosure is to provide a food composition capable of effectively preventing or alleviating a muscular disease.

A further aspect of the present disclosure is to provide a health functional food capable of effectively preventing or alleviating a muscular disease.

A still further aspect of the present disclosure is to provide a feed additive composition capable of effectively preventing or alleviating a muscular disease.

### Solution to Problem

To accomplish above purposes,

The present disclosure provides a food composition including a mealworm protein isolate or a hydrolysate thereof as an active ingredient for prevention or alleviation of a muscular disease.

In an embodiment, the mealworm larva may be prepared by a process including the steps of: a) pulverizing a dried mealworm larva mass; b) defat the pulverized mass by addition of ethanol thereto; c) adding and mixing the defatted mealworm larva mass with sodium hydroxide, followed by centrifugation to obtain a pellet; and d) desalting the pellet, followed by lyophilization.

In an embodiment, the hydrolysate may be prepared by treating the mealworm protein isolate with an alcalase, flavourzyme, or a combination thereof.

In an embodiment of the present disclosure, the mealworm protein isolate or the hydrolysate thereof can inhibit the expression of myostatin.

In an embodiment of the present disclosure, the muscular disease may be a muscular disease caused by muscle dysfunction, muscle loss, muscle atrophy, muscle wasting, or muscle degeneration.

In an embodiment of the present disclosure, the muscular disease may be selected from the group consisting of atony, muscular atrophy, muscular dystrophy, myasthenia, cachexia, rigid spine syndrome, amyotrophic lateral sclerosis (Lou Gehrig's disease), Charcot-Marie-Tooth disease, sarcopenia, and a combination thereof.

In addition, the present disclosure provides a health functional food including a mealworm protein isolate or a hydrolysate thereof as an active ingredient for prevention or alleviation of a muscular disease.

In an embodiment of the present disclosure, the health functional food may be selected from the group consisting of beverages, meats, confectionaries, noodles, rice cakes, breads, gums, candies, ice creams, and liquors.

Furthermore, the present disclosure provides a pharmaceutical composition including a mealworm protein isolate or a hydrolysate thereof as an active ingredient for prevention or treatment of a muscular disease.

Moreover, the present disclosure provides a feed additive composition including a mealworm protein isolate or a hydrolysate thereof as an active ingredient for prevention or alleviation of a muscular disease.

### Advantageous Effects of Invention

The mealworm protein isolate or the hydrolysate thereof according to the present disclosure can effectively inhibit the expression of myostatin, so that a composition containing same as an active ingredient for prevention, alleviation, or treatment of various muscular diseases caused by muscle dysfunction, muscle loss, muscle atrophy, muscle wasting, and muscle degeneration, finding advantageous applications in the medicinal product, food, and feedstuff industries.

### BRIEF DESCRIPTION OF DRAWINGS

FIG. 1 is a flow chart illustrating processes of preparing mealworm protein isolates (MPI) and hydrolysates thereof (MPH) as conducted in Examples 1 and 2 according to the present disclosure.

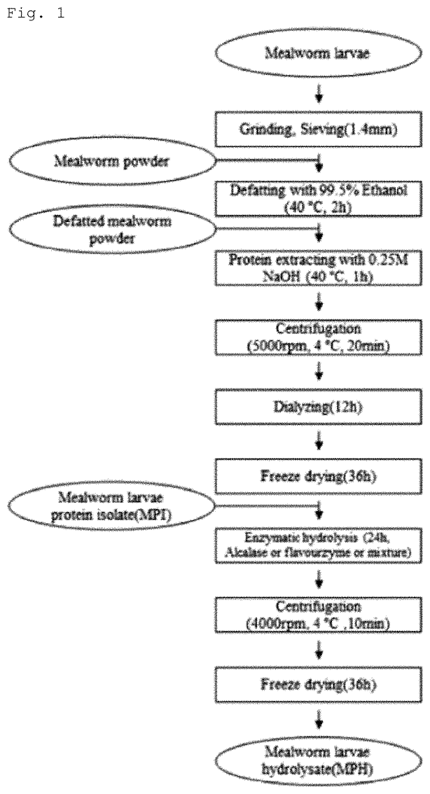

FIG. 2 shows cytotoxicity assay results of the mealworm protein isolates (MPI) and hydrolysates thereof (MPHAF, AF-LT, AF-TT, and AF-MT) according to the present disclosure.

FIG. 3 shows relative mRNA expression levels of myostatin in C2C12 cells treated with 0.01 mg/mL of each of the mealworm protein hydrolysates (MPHAF, MPHFA, MPHME, MPHA, and MPHF) prepared under different hydrolysis conditions set forth according to the present disclosure.

FIG. 4 shows relative mRNA expression levels of myostatin in C2C12 cells after treatment with 0.01 mg/mL of each hydrolysate (AF-LT, AF-TT, AF-MT) prepared according to size from MPHAF.

FIG. 5 shows relative myostatin promoter activity in C2C12 cells after treatment with 0.01 mg/mL of each of the mealworm protein isolate (MPI) and hydrolysate thereof (MPH) according to the present disclosure.

FIG. 6 shows expression levels of inflammatory cytokines (IL-6, TNF-α, and IL-1b) in LPS-treated macrophages after treatment with mealworm protein hydrolysates (MPHAF, MPHFA, MPHME, MPHA, MPHF) prepared under different hydrolysis conditions set forth according to the present disclosure.

FIG. 7 shows expression levels of inflammatory cytokines (IL-6, TNF-α, and IL-1b) in LPS-treated macrophages after treatment with the hydrolysates (AFLT, AFTT, and AFMT) prepared according to size from MPHAF.

FIG. 8 shows levels of available amino groups in the mealworm protein hydrolysates (MPHAF, MPHFA, MPHME, MPHA, and MPHF) prepared under different hydrolysis conditions set forth according to the present disclosure.

FIG. 9 shows protein levels of the mealworm protein hydrolysates (MPHAF, MPHFA, MPHME, MPHA, and MPHF) prepared under different hydrolysis conditions set forth according to the present disclosure.

FIG. 10 shows protein levels of the hydrolysates (AF-LT, AF-TT, and AF-MT) prepared according to size from MPHAF.

FIG. 11 shows SDS-PAGE patterns of the mealworm protein hydrates prepared under different hydrolysis conditions set forth according to the present disclosure.

FIG. 12 shows anti-oxidative activity assay results of the mealworm protein hydrolysates (MPHAF, MPHFA, MPHME, MPHA, and MPHF) prepared under different hydrolysis conditions set forth according to the present disclosure.

FIG. 13 shows anti-oxidative activity assay results of the hydrolysates (AF-LT, AF-TT, and AF-MT) prepared according to size from MPHAF.

### BEST MODE FOR CARRYING OUT THE INVENTION

The present disclosure relates to a pharmaceutical composition including a mealworm protein isolate or a hydrolysate thereof as an active ingredient for prevention or treatment of a muscular disease.

As used herein, the term “prevention” refers to all actions of inhibiting or delaying the onset of a muscular disease by administration of the composition according to the present disclosure.

As used herein, the term “treatment” refers to all actions involved in alleviating or beneficially changing symptoms of a muscular disease by administration of the composition according to the present disclosure.

The term “mealworm protein isolate”, as used herein, refers to a protein extracted from larvae of mealworms.

The mealworm protein isolate may be prepared by a process including the steps of: a) pulverizing a dried mealworm larva mass; b) defat the pulverized mass by addition of ethanol thereto; c) adding and mixing the defatted mealworm larva mass with sodium hydroxide, followed by centrifugation to obtain a pellet; and d) desalting the pellet, followed by lyophilization.

The “mealworm protein hydrolysate”, as used herein, refers to a substance prepared by hydrolyzing the mealworm protein isolate with a hydrolysis enzyme.

The hydrolysate may be prepared by treating the mealworm protein isolate with a hydrolysis enzyme selected from alcalase, flavourzyme, and a combination thereof. When used in combination, alcalase and flavourzyme may be applied to the mealworm protein isolate in that order or in the reverse order.

In an embodiment of the present disclosure, the hydrolysate may be obtained by treating the mealworm protein isolate with 0.5% (w/v) alcalase for 12 hours and then with 0.5% (w/v) flavourzyme for an additional 12 hours.

In another embodiment of the present disclosure, the hydrolysate may be obtained by treating the mealworm protein isolate with 0.5% (w/v) alcalase for 12 hours and then with 0.5% (w/v) flavourzyme for an additional 12 hours and may have a molecular weight of 10 KDa or greater.

The mealworm protein isolate or hydrolysate thereof according to the present disclosure may exhibit a prophylactic or therapeutic effect on a muscular disease by inhibiting the expression of myostatin.

As used herein, the term “myostatin” (MSTN) refers to a protein, also known as growth differentiation factor-8 (GDF-8), which belongs to the transforming growth factor-β (TGF-β) family, functioning to regulate muscle growth.

As used herein, the term “muscular disease” refers to a disease caused by muscle dysfunction, muscle loss, muscle atrophy, muscle wasting, or muscle degeneration.

The term “muscle” as used herein collectively refers to tendon, muscle, and sinew, and “muscle function” means the ability of muscle to exert a force through muscle contraction, and encompasses: muscle strength, which is the ability of muscle to exert maximum contraction to overcome resistance; muscular endurance, which is the ability to indicate how long or how many times muscle can repeat contraction and relaxation at a given weight; and explosive muscular strength, which is the ability to exert a strong force within a short period of time. Such muscle functions are proportional to muscle mass, and “muscle function improvement” means the act of making muscle functions better.

In an embodiment of the present disclosure, the muscular disease is preferably at least one selected from the group consisting of atony, muscular atrophy, muscular dystrophy, myasthenia, cachexia, rigid spine syndrome, amyotrophic lateral sclerosis (Lou Gehrig's disease), Charcot-Marie-Tooth disease, sarcopenia, and a combination thereof, but with no limitations thereto. In addition, the muscle wasting or the muscle degeneration occurs due to genetic factors, acquired factors, aging, or the like. The muscle wasting is characterized by the gradual loss of muscle mass and the weakness and degeneration of muscles, especially skeletal or voluntary muscles and cardiac muscles.

In an embodiment of the present disclosure, the mealworm protein isolate or the hydrolysate thereof contained in the composition may be used in an amount of 0.001% by weight to 99% by weight and more particularly in an amount of 0.01% by weight to 50% by weight, based on the total weight of the composition, but with no limitations thereto. Meanwhile, the mealworm protein isolate or the hydrolysate thereof according to the present disclosure may be contained at a concentration of 0.0001 to 1000 μg/ml in the composition.

The composition of the present disclosure may be used as a pharmaceutical composition containing the mealworm protein isolate or the hydrolysate thereof as an active ingredient. In addition to the active ingredient, the pharmaceutical composition may contain a biologically acceptable auxiliary agent which may be exemplified by an excipient, a disintegrant, a sweetener, a binder, a coating agent, a swelling agent, a lubricant, a slip modifier, a flavorant, and the like.

For administration, the pharmaceutically composition may be formulated along with at least one pharmaceutically acceptable vehicle in addition to the active ingredient.

The pharmaceutical composition of the present disclosure may be in various oral or parenteral preparations. When the composition is prepared into a formulation, use may be made of at least one of buffers (e.g., saline or PBS), antioxidants, bacteriostatic agents, chelating agents (e.g., EDTA or glutathione), fillers, extenders, binders, adjuvants (e.g., aluminum hydroxide), suspending agents, thickeners, wetting agents, disintegrants or surfactants, and diluents or excipients.

Solid preparations for oral administration include tablets, pills, powders, granules, capsules, and the like. Such solid preparations may be prepared by mixing one or more compounds with at least one excipient such as starch (including corn starch, wheat starch, rice starch, potato starch, and the like), calcium carbonate, sucrose, lactose, dextrose, sorbitol, mannitol, xylitol, erythritol, maltitol, cellulose, methyl cellulose, sodium carboxymethyl cellulose, hydroxypropyl methylcellulose, and gelatin. For example, tablets or sugarcoated tablets may be obtained by blending an active ingredient with a solid excipient, grinding the blend, adding a suitable adjuvant thereto, and then processing the resultant into a granule mixture.

In addition to simple excipients, lubricants such as magnesium stearate and talc are also used. Liquid preparations for oral administration include suspensions, solutions, emulsions, syrups, and the like. In addition to water or liquid paraffin, which is a commonly used simple diluent, the liquid preparations may contain various excipients such as a wetting agent, a sweetener, a fragrance, and a preservative. In addition, in some cases, crosslinked polyvinylpyrrolidone, agar, alginic acid, sodium alginate, or the like may be added as a disintegrant. An anticoagulant, a lubricant, a wetting agent, a flavoring agent, an emulsifying agent, an antiseptic agent, or the like may be further contained.

Preparations for parenteral administration include sterile aqueous solutions, non-aqueous solvents, suspensions, emulsions, lyophilized preparations, and suppositories. For the non-aqueous solvents and suspensions, propylene glycol, polyethylene glycol, vegetable oil such as olive oil, injectable ester such as ethyl oleate, and the like can be used. As bases of the suppository, witepsol, macrogol, Tween 61, cacao butter, laurin butter, glycerol, gelatin, or the like may be used.

The pharmaceutical composition of the present disclosure may be administered orally or parenterally. For parenteral administration, the pharmaceutical composition may be formulated, according to methods known in the art, into a skin agent for external use; an injection to be injected intraperitoneally, rectally, intravenously, intramuscularly, subcutaneously, intrauterine epidurally, or intracerebrovascularly; a preparation for transdermal administration; or a nasal inhalant.

The injection must be sterilized and protected against contamination of microorganisms such as bacteria and fungi. In a case of the injection, examples of suitable carriers may include, but are not limited to, solvents or dispersion media which contain water, ethanol, polyol (for example, glycerol, propylene glycol, and liquid polyethylene glycol), mixtures thereof, and/or vegetable oil. More preferably, an isotonic solution such as Hank's solution, Ringer's solution, triethanolamine-containing phosphate buffered saline (PBS) or sterilized water for injection, 10% ethanol, 40% propylene glycol, and 5% dextrose, or the like may be used as a suitable carrier. In order to protect the injection against contamination of microorganisms, various antibacterial agents and antifungal agents such as paraben, chlorobutanol, phenol, sorbic acid, and thimerosal may be further contained. In addition, in most cases, the injection may further contain an isotonic agent such as sugar or sodium chloride.

The preparation for transdermal administration takes forms such as an ointment, a cream, a lotion, a gel, a liquid for external use, a paste, a liniment, and an aerosol. In this regard, “transdermal administration” means administering a pharmaceutical composition topically to skin so that an effective amount of an active ingredient contained in the pharmaceutical composition is delivered into the skin.

In a case of a preparation for inhalation administration, the active ingredient to be used according to the present disclosure may be conveniently delivered in the form of an aerosol spray from a pressurized pack or a nebulizer, using a suitable propellant such as dichlorofluoromethane, trichlorofluoromethane, dichlorotetrafluoroethane, carbon dioxide, or another suitable gas. For a pressurized aerosol, a unit dosage may be determined by providing a valve that delivers a metered amount. By way of example, gelatin capsules and cartridges for use in an inhaler or insufflator may be formulated to contain a powder mixture of a compound and a suitable powder base such as lactose and starch. Preparations for parenteral administration are described in Remington's Pharmaceutical Science, 15th Edition, 1975. Mack Publishing Company, Easton, Pa. 18042, Chapter 87: Blaug, Seymour, which is a prescription manual commonly known in all pharmaceutical chemistries.

The pharmaceutical composition of the present disclosure is administered in a pharmaceutically effective amount. In the present disclosure, the term “pharmaceutically effective amount” means an amount which is sufficient to treat a disease at a reasonable benefit/risk ratio applicable to medical treatment, and an effective dose level can be determined depending on factors including type, severity of the patient's disease, activity of the drug, sensitivity to the drug, time of administration, route of administration and rate of excretion, duration of treatment, the simultaneously used drug, and other factors well known in the medical field. The pharmaceutical composition of the present disclosure may be administered as an individual therapeutic agent or in combination with another therapeutic agent.

The pharmaceutical composition may be administered alone as an individual therapeutic or in combination with another therapeutic agent and may be administered sequentially or simultaneously with a conventional therapeutic agent, and may be administered in single or multiple doses. That is, a total effective amount of the pharmaceutical composition of the present disclosure may be administered to a patient as a single dose, or as multiple doses by a fractionated treatment protocol intended for a long-term administration. It is important to administer an amount such that a maximum effect can be obtained with a minimum amount without side effects by taking all of the above-described factors into consideration, and such an amount can be readily determined by those skilled in the art.

The dose of the pharmaceutical composition of the present disclosure varies depending on the patient's body weight, age, sex, health condition, diet, time of administration, mode of administration, excretion rate, and severity of the disease.

The pharmaceutical composition of the present disclosure may be used either alone or in combination with methods which use surgery, radiation therapy, hormonal therapy, chemotherapy, or biological response modifiers.

The pharmaceutical composition of the present disclosure may also be provided as a preparation for external use comprising mealworm protein isolate or a hydrolysate thereof as an active ingredient. In a case where the pharmaceutical composition of the present disclosure for preventing or treating a muscular disease is used as a skin agent for external use, the pharmaceutical composition may further contain adjuvants commonly used in the field of dermatology such as any other ingredients commonly used for skin agent for external use including a fatty substance, an organic solvent, a solubilizing agent, a concentrating agent and a gelling agent, a softening agent, an antioxidant, a suspending agent, a stabilizing agent, a foaming agent, a fragrance, a surfactant, water, an ionic emulsifying agent, a nonionic emulsifying agent, a filling agent, a metal ion blocking agent, a chelating agent, a preservative, a vitamin, a blocking agent, a wetting agent, essential oil, a dye, a pigment, a hydrophilic activator, a lipophilic activator, a lipid vesicle, and the like. In addition, the above ingredients may be introduced in an amount commonly used in the field of dermatology.

When provided as a skin agent for external use, the pharmaceutical composition for preventing or treating a muscular disease according to the present disclosure may be, but is not limited to, a formulation such as an ointment, a patch, a gel, a cream, and a spray.

In addition, the present disclosure relates to a use of a composition including a mealworm protein isolate or hydrolysate thereof as an active ingredient for preventing or treating a muscular disease. The composition including a mealworm protein isolate or a hydrolysate thereof as an active ingredient according to the present disclosure may be used in preparing a medication for preventing or a treating a muscular disease.

Also, the present disclosure is drawn to a method for preventing or treating a muscular disease, the method comprising treating a mealworm protein isolate or hydrolysate thereof at a therapeutically effective dose to a mammal.

As used herein, the term “mammal” refers to a mammalian subject to be treated, observed, or tested and particularly to a human.

As used herein, the term “therapeutically effective amount” refers to an amount of an active ingredient or pharmaceutical composition that will elicit the biological or medical response of a tissue system, animal or human, as considered by a researcher, veterinarian, medical practitioner or clinician, and is intended to encompass an amount of the active ingredient or pharmaceutical composition which will ameliorate the symptoms of the disease or disorder being treated. As will be apparent to those skilled in the art, the therapeutically effective dose and administration times of the active ingredient in accordance with the present disclosure may vary depending upon desired therapeutic effects. Therefore, an optimal dose of the active drug to be administered can be easily determined by those skilled in the art and may vary depending on various factors including kinds of disease, severity of disease, contents of active ingredients and other components contained in the composition, kinds of formulations, age, weight, general health status, sex and dietary habits of patients, administration times and routes, release rates of the composition, treatment duration, and co-administered drugs. In the treatment method of the present disclosure, the mealworm protein isolate or hydrolysate thereof according to the present disclosure may be administered once or several times a day at a dose of 0.0001 mg/kg-1000 mg/kg for adults, but with no limitations thereto.

In the treatment method of the present disclosure, the composition according to the present disclosure can be administered in a typical manner through oral, rectal, intravenous, intraarterial, intraperitoneal, intramuscular, intrasternal, transdermal, topical, intraocular, or intradermal routes.

According to the present disclosure, the pharmaceutical composition containing a mealworm protein isolate or a hydrolysate thereof may be formulated together with or may be used in combination with a conventional therapeutic agent for muscular disease.

Moreover, the present disclosure provides a food composition containing a mealworm protein isolate or a hydrolysate thereof as an active ingredient.

The food composition may further contain an additional ingredient such as a flavoring agent or a natural carbohydrate in addition to a mealworm protein isolate or a hydrolysate thereof as an active ingredient.

Examples of the natural carbohydrate include monosaccharides such as glucose and fructose; disaccharides such as maltose and sucrose; polysaccharides such as dextrin and cyclodextrin; and sugar alcohols such as xylitol, sorbitol, and erythritol. As the flavoring agent, a natural flavoring agent (thaumatin), a Stevia extract (e.g., rebaudioside A, glycyrrhizin, etc.), and a synthetic flavoring agent (saccharin, aspartame, etc.) may be advantageously used.

The food composition of the present disclosure may be formulated in the same manner as for the pharmaceutical composition and may be used as a functional food or added to various foods. Examples of the foods to which the composition of the present disclosure may be added include beverages, meats, chocolates, foods, snacks, pizzas, ramens, other noodles, gums, candies, ice creams, alcoholic beverages, vitamin complexes, and health supplement foods.

Further, the food composition may include various nutrients, vitamins, minerals (electrolytes), flavors such as synthetic flavors and natural flavors, coloring agents, and enhancers (cheese, chocolate, etc.), pectic acid and its salts, alginic acid and its salts, organic acids, protective colloid thickeners, pH adjusting agents, stabilizers, preservatives, glycerin, alcohols, carbonating agents used in carbonated beverages, and the like in addition to the active ingredient (mealworm protein isolate or hydrolysate thereof). In addition, the food composition of the present disclosure may include flesh for use in producing natural fruit juices, fruit juice drinks, and vegetable drinks.

As a natural material which is almost free of side effects such as in chemicals, the active ingredient (mealworm protein isolate or hydrolysate thereof) of the present disclosure can be safely used for a long period of time to impart the function of alleviating a muscular disease.

That is, the food composition of the present disclosure can be advantageously used as a functional food composition for prevention or alleviation of a muscular disease.

Also, the present disclosure provides a health functional food including a mealworm protein isolate or a hydrolysate thereof as an active ingredient for prevention or alleviation of a muscular disease.

For the purpose of preventing or alleviating a muscular disease, the health functional food of the present disclosure may be prepared or processed into the form of tablets, capsules, powders, granules, liquids, pills, etc.

As used herein, the term “health functional food” refers to a food that is produced or processed using a raw material or component having useful functionality in the human body in accordance with Act 6727 on the Health Functional Food and which is ingested to acquire a beneficial effect for health use such as controlling nutrients or physiological action for the structure and function of the human body.

The health functional food of the present disclosure may include common food additives. So long as there are no other regulations, the conformity of the “food additive” is determined in consideration with the standard and criteria of the corresponding item according to the general rule of the food additives codex and general tests approved by the Korean Ministry of Food and Drug Safety.

The items listed on the “food additives codex” include chemical compounds such as ketones, glycine, calcium citrate, nicotinic acid, and cinnamic acid;

natural additives such as persimmon color, licorice extract, crystalline cellulose, kaoliang color, and guar gum; and mixed preparations such as L-glutamic acid sodium preparations, alkalis additives for noodle, preservative preparations, and tar color preparations.

For example, in order to produce a health functional food in a tablet form, a mixture of the active ingredient (mealworm protein isolate or hydrolysate thereof) of the present disclosure, an excipient, a binder, a disintegrant, and other additives can be granulated using a conventional method, and then compression molding process is performed with lubricants. Alternatively, the mixture can be directly subjected to the compression molding process. In addition, when needed, the health functional food in a tablet form may include sweeteners.

Among health functional foods in a capsule form, a hard capsule formulation can be produced by filling a conventional hard capsule with a mixture of the active ingredient mealworm protein isolate or hydrolysate thereof) of the present disclosure and additives such as excipients; and a soft capsule formulation can be produced by filling a capsule support of gelatin with a mixture of th the active ingredient (mealworm protein isolate or hydrolysate thereof) of the present disclosure, and additives such as excipients. The soft capsule formulation may include plasticizers, such as glycerin or sorbitol, coloring agents, and preservatives, as needed.

A health functional food in a pill form can be produced by molding a mixture of the active ingredient (mealworm protein isolate or hydrolysate thereof) of the present disclosure, excipients, binders, and disintegrants using well-known methods. The pills may be coated with white sugar or other coating materials, or the surface thereof can be coated with starch, talc, or other materials, as necessary.

A health functional food in a granule form can be produced by granulating a mixture of the active ingredient (mealworm protein isolate or hydrolysate thereof) of the present disclosure, excipients, binders, and disintegrants using well-known methods. When needed, the health functional food in a granule form can include flavoring agents and sweeteners.

The health function food including a mealworm protein isolate or a hydrolysate thereof as an active ingredient according to the present disclosure exhibits an inhibitory effect on the expression of myostatin as proved in the following Examples and thus is effective for preventing or alleviating muscular diseases such as atony, muscular atrophy, muscular dystrophy, myasthenia, cachexia, rigid spine syndrome, amyotrophic lateral sclerosis (Lou Gehrig's disease), Charcot-Marie-Tooth disease, and sarcopenia.

The health functional food may be beverages, meats, chocolates, foods, snacks, pizza, ramens, other noodles, gums, candies, ice creams, alcoholic beverages, vitamin complexes, and health supplement foods.

In addition, the present disclosure provides a cosmetic composition including a mealworm protein isolate or a hydrolysate thereof as an active ingredient for prevention or alleviation of a muscular disease. The cosmetic composition may be externally applied to the skin or orally ingested, but with no limitations thereto.

The cosmetic composition of the present disclosure contains a mealworm protein isolate or hydrolysate thereof as an active ingredient and may be prepared, together with a dermatologically acceptable excipient, into the form of basic cosmetics (toner, cream, essence, cleanser such as cleansing foam or cleansing water, pack, and body oil), color cosmetics (foundation, lipstick, mascara, and makeup base), hair products (shampoo, rinse, hair conditioner, and hair gel), and soap.

The above dermatologically acceptable excipients may include, but are not limited to, a skin softener, a skin infiltration enhancer, a colorant, an aromatic, an emulsifier, a thickener, or a solvent. In addition, it is possible to add fragrance, a pigment, bactericidal agent, an antioxidant, a preservative, moisturizer, and the like, while adding thickening agents, inorganic salts, or synthetic polymers for improving physical properties. For example, in case of manufacturing a cleanser and a soap containing the cosmetic composition, they may be prepared by adding a mealworm protein isolate or a hydrolysate thereof to a conventional cleanser and soap base. For a cream, it may be prepared by adding a mealworm protein isolate or a hydrolysate thereof to conventional oil-in-water cream base. Furthermore, it is possible to add a fragrance, a chelating agent, a pigment, an antioxidant, a preservative, and the like, and to add synthetic or natural materials such as proteins, minerals, or vitamins, for improving physical properties.

Also contemplated according to the present disclosure is a feed additive composition containing a mealworm protein isolate or a hydrolysate thereof as an active ingredient for prevention or treatment of a muscular disease.

In detail, the mealworm protein isolate or the hydrolysate thereof according to the present disclosure exhibits the effect of increasing overall muscular strength through an increase in skeletal musculoskeletal strength and an improvement in muscle function and thus can be contained as a growth enhancer in a feed additive for animals and livestock.

The “feed additive” refers to a substance added to the feed for various effects of supplementing nutrients and preventing weight loss, improving digestibility of fiber in the feed, improving milk quality, preventing reproductive disorders and improving fertility, and preventing high temperature stress in the summer. The feed additive of the present disclosure is supplement feed according to the Control of Livestock and Fish Feed Act, and mineral preparations such as sodium hydrogen carbonate, bentonite, magnesium oxide and complex minerals, mineral preparations like trace minerals such as zinc, copper, cobalt and selenium, vitamins such as keratin, vitamin E, vitamins A, D and E, nicotinic acid and vitamin B complex, protective amino acids such as methionine and lysine, protected fatty acids such as fatty acid calcium salts, probiotics (lactic acid bacteria), yeast cultures, live bacteria such as fungal fermented products, yeast preparations, and the like can be additionally included.

No particular limitations are imparted to the subject to which the feed additive composition of the present disclosure can be applied, as long as it is intended to generally increase in muscular strength and growth through the increase of skeletal muscle mass or the improvement of muscle functions and bone density. Examples of the subject include mammals including non-primates (e.g., cattle, pigs, horses, cats, dogs, rats, and mice) and primates (e.g., monkeys, i.e., cynomolgous monkeys and chimpanzees). In another embodiment, examples of the subject include livestock (e.g., horses, cattle, pigs, and the like) or companion animals (e.g., dogs or cats).

### MODE FOR CARRYING OUT THE INVENTION

Below, a better understanding of the present disclosure may be obtained through the Examples which are set forth to illustrate, but are not to be construed to limit the present disclosure.

### Example 1

Extraction of Mealworm Protein Isolate

In this experiment, a protein was extracted from mealworm larvae. The extracted protein was named mealworm protein isolate, abbreviated as “MPI”.

<1-1>Raw Material of Mealworm Larva

Mealworm larvae were purchased as dried mass from Edible Bug Co., Seoul, Korea. After pulverization, the dried mealworm larvae were let to pass through a 1.4-mm sieve to afford a mealworm larva powder which was then stored at 4° C. until use.

<1-2>Defatting Mealworm Larva

A defatting process increased efficiency of protein extraction from mealworm larvae. That is, mealworm larvae were added at a ratio of 1:5 with 99.5% ethanol, followed by extraction at 40° C. for 60 minutes in a shaking bath (VS-1205 SW1, Vision Scientific Co., Ltd, Daejeon, Korea). This procedure was repeated twice before evaporation of ethanol for 12 hours.

<1-3>Extraction Process for Mealworm Protein Isolate

After 0.25M NaOH was added at a rate of 1:15 to the defatted mealworm larva, mixing was carried out 40° C. for 60 minutes using a hot plate & magnetic stirrer (Vision Science Co., Korea). A supernatant was obtained by centrifugation at 4° C. and 4200 rpm for 15 minutes in a centrifuge (VS-24SMTi, Vision Science Co., Ltd, Korea) and adjusted into a pH of 4.5. Subsequently, additional centrifugation at 4° C. and 4200 rpm for 15 minutes afforded an extract as a pellet.

<1-4>Dialysis and Lyophilization of Pellet

The pellet was desalted for 12 hours in a dialysis bag (12 KDa MWCO; Sigma-Aldrich Chemical Co., St. Louis, Mo., USA). Subsequently, lyophilization for 36 hours afforded the desired mealworm protein isolate (MPI) of the present disclosure.

### Example 2

Hydrolysate of Mealworm Protein Isolate

In this experiment, a hydrolysate of the mealworm protein isolate was prepared using a protease. The hydrolysate of the mealworm protein isolate was named mealworm protein hydrolysate, abbreviated as “MPH”.

<2-1>Raw Material of Mealworm Protein Isolate

The mealworm protein isolate (MPI) prepared in Example 1 was used as a raw material.

<2-2>Hydrolysis Condition of Mealworm Protein Isolate

With reference to previous studies on substrate concentrations, enzyme concentrations, pH, reaction temperatures, and reaction times, an optimal condition for hydrolysis of the mealworm protein isolate was established as follows: substrate concentration (1%), enzyme concentration (alcalase, flavourzyme, alcalase+flavourzyme 1%), pH (8), reaction temperature (55° C.), and reaction time (12 hours or 24 hours)

<2-3>Inactivation of Intrinsic Enzyme of Mealworm Protein Isolate

The protein powder was dispersed at the established substrate concentration in a buffer with a pH of 8 and incubated at 85° C. for 20 minutes in a water bath to inactivate its intrinsic enzymes.

<2-4>Establishment of pH Environment for Hydrolysis of Mealworm Protein Isolate

The intrinsic enzyme-inactivated protein solution was transferred to different water baths having reaction temperatures set to respective conditions and cooled to the temperatures, followed by adjusting its pH into predetermined values with 1 N NaOH.

<2-5>Acquirement of Mealworm Protein Hydrolysate

The mealworm protein isolate was treated with 1% (w/v) of each enzyme (alcalase (Novozymes A/S. Co., Ltd., Bagsvaerd, Denmark), and flavourzyme) to conduct hydrolysis, with its pH maintained at 8. Enzyme treatment and reaction time are summarized in Table 1, below. Following centrifugation, the hydrolysate was obtained as the supernatant. It was then lyophilized for 36 hours to acquire the final mealworm protein isolate of the present disclosure.
MPHA: hydrolysate obtained after treatment with 1% (w/v) alcalase for 24 hours
MPHF: hydrolysate obtained after treatment with 1% (w/v) flavourzyme for 24 hours
MPHME: hydrolysate obtained after treatment with mixed enzymes (0.5% (w/v) alcalase & 0.5% (w/v) flavourzyme for 24 hours
MPHAF: hydrolysate obtained after treatment with 0.5% (w/v) alcalase for 12 hours and then with 0.5% (w/v) flavourzyme for 12 hours
MPHFA: hydrolysate obtained after treatment with 0.5% (w/v) flavourzyme for 12 hours and then with 0.5% (w/v) alcalase for 12 hours

TABLE 1
Enzymatic Treatment Method of Mealworm
Protein Hydrolysate and Reaction Time

| | Enzymatic Treatment Condition |
|---|---|
| Hydrolysate | (% (w/v) relative to substrate, |
| name | enzyme type, reaction time) |
| MPHA | (1% (w/v), alcalase, 24 h) |
| MPHF | (1% (w/v), flavourzyme, 24 h) |
| MPHME | (0.5% (w/v) alcalase + |
| | 0.5% (w/v) flavourzyme, 24 h) |
| MPHAF | (0.5% (w/v), alcalase, 12 h) → |
| | (0.5% (w/v), flavourzyme, 12 h) |
| MPHFA | (0.5% (w/v), flavourzyme,12 h) → |
| | (0.5% (w/v), alcalase, 12 h) |

<2-6>MPHAF Acquirement by Size

The hydrolysate obtained under the MPHAF condition was centrifuged at Rcf 4200 for 25 minutes using Pierce™ Protein Concentrator PES, 10K MWCO (Thermo Scientific™, MA, USA). Hydrolysates with a molecular weight of greater than 10 kDa were obtained from the lower layer and named “AF-MT”. the upper layer was further centrifuged using Pierce™ Protein Concentrator PES, 3K MWCO (Thermo Scientific™, MA, USA) to divide hydrolysates ranging in molecular weight from 3 kDa to 10 kDa and hydrolysates with a molecular weight less than 3 kDA.
AF-LT: MPHAF with a molecular weight of less than 3 kDa
AF-TT: MPHAF with a molecular weight of 3 to 10 kDa
AF-MT: MPHAF with a molecular weight of more than 10 kDa

### Experimental Example 1

Effect on Myostatin Expression

<1-1>Cytotoxicity Assay (MTT Assay)

Prior to examination of myostatin expression levels, the sample was measured for cytotoxicity by MTT assay.

The myoblast cell line C2C12 mouse normal cells were incubated for 24 hours in DEME. When the cells grew to confluence, the medium was aspirated and trypsin was added to detach the cells from the petri dish. After 10% FBS was added, the cells were harvested as a pellet by centrifugation. Then, 10 μl of trypan blue was added to the cells which were then loaded on a cell counting slide and counted using an automated cell counting system. Following counting entire cells, a predetermined number of cells was mixed with a medium, seeded into well plates, and incubated at 37° C. for 24 hours in an incubator. Thereafter, the medium for the C2C12 cells was replaced with 1 mL of a fresh one. The samples (WPI, MPI, MPHAF, AF-LT, AF-TT, AF-MT) were serially diluted into concentrations of 1 mg/mL, 0.1 mg/mL, and 0.01 mg/mL in distilled water and added to the medium before being put into a 37° C. incubator. After 24 hours of incubation at 37° C., the C2C12 cells were withdrawn from the incubator and the medium was removed. MTT reagent was added in an amount of 10 μl to each well and incubated at 37° C. for 2 hours in an incubator. Then, the medium and MTT reagent were aspirated from each well. After addition of 50 μl of DMSO, living cells were counted using a Microplate Spectrophotometer (Epoch, BioTek).

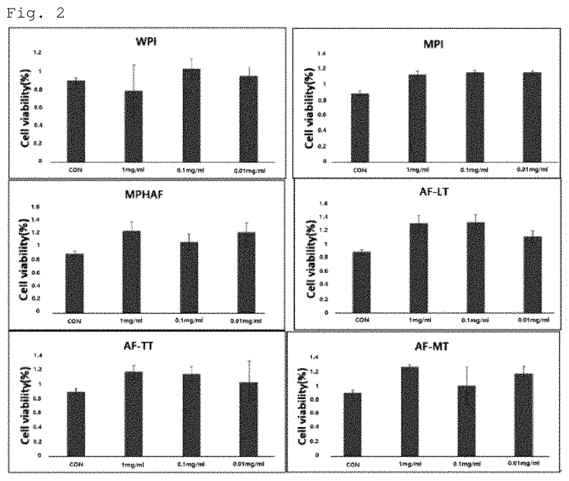

The results are shown in FIG. 2 and Table 2. Except for 10 mg/ml WPI, as can be seen, all samples (MPI, MPHAF, AF-LT, AF-TT, and AF-MT) exhibited higher cell viability at every concentration than the controls.

TABLE 2
Assay for Cytotoxicity of Mealworm
Protein Hydrolysate

| | Treatment | | | Standard |
|---|---|---|---|---|
| Sample | conc. | | Mean | deviation |
| WPI | con | | 0.898 | 0.035 |
| | 1 | mg/ml | 0.787 | 0.282 |
| | 0.1 | mg/ml | 1.026 | 0.114 |
| | 0.01 | mg/ml | 0.95 | 0.089 |
| MPI | con | | 0.898 | 0.035 |
| | 1 | mg/ml | 1.142 | 0.048 |
| | 0.1 | mg/ml | 1.167 | 0.027 |
| | 0.01 | mg/ml | 1.168 | 0.021 |
| MPHAF | con | | 0.898 | 0.035 |
| | 1 | mg/ml | 1.239 | 0.134 |
| | 0.1 | mg/ml | 1.068 | 0.122 |
| | 0.01 | mg/ml | 1.218 | 0.143 |
| AF-LT | con | | 0.898 | 0.035 |
| | 1 | mg/ml | 1.32 | 0.115 |
| | 0.1 | mg/ml | 1.331 | 0.116 |
| | 0.01 | mg/ml | 1.123 | 0.088 |
| AF-TT | con | | 0.898 | 0.035 |
| | 1 | mg/ml | 1.172 | 0.087 |
| | 0.1 | mg/ml | 1.14 | 0.103 |
| | 0.01 | mg/ml | 1.031 | 0.29 |
| AF-MT | con | | 0.898 | 0.035 |
| | 1 | mg/ml | 1.263 | 0.026 |
| | 0.1 | mg/ml | 0.994 | 0.267 |
| | 0.01 | mg/ml | 1.166 | 0.099 |

<1-2>Relative mRNA Expression Level of Myostatin

To examine hydrolysis conditions of the mealworm protein isolates and sizes of the hydrolysates for prophylactic and palliative effects on sarcopenia, myostatin expression levels were measured. In this regard, whey protein isolate (WPI) purchased from (PUREUNBIN Co., Yeongcheon-si, Gyeongsangbuk-do, Korea) was used as a control.

After examination was made of the concentrations at which the samples did not exert cytotoxic activity in Experimental Example <1-1>, C2C12 cells were incubated at 37° C. for 24 hours. Thereafter, the medium of the C2C12 cells was replaced by 1 mL of a fresh one. Afterward, 1 μl of distilled water containing the sample (MPI, MPH) at a concentration of 0.01 mg/mL sample was added to each well (PBS added for the control), followed by incubation at 37° C. for 24 hours. Subsequently, the medium was aspirated and the cells were washed twice with PBS. Then, 200 μl of TRIzol was added to each well. The cells in each well were left at room temperature for 5 minutes and mixed with 40 μl of chloroform. Following centrifugation at 13,200 rpm and 4° C. for 15 minutes, the supernatant thus obtained was mixed with 120 μl of isopropanol and left at room temperature for 10 minutes. Again, centrifugation was carried out at 13,200 rpm and 4° C. for 15 minutes, and the supernatant was removed. Addition of 100 μl of 70% chilled ethanol was followed by centrifugation at 13,200 rpm and 4° C. for 2 minutes. This process was repeated twice. Subsequently, the 70% ethanol was removed and DEPC (diethyl pyrocarbonate)-water was added. RNA concentrations were measured using a microplate spectrophotometer (Epoch, BioTek). According to the measurements of RNA concentrations in the samples, the samples and DEPC-water were additionally added and mixed with 10 μl of TOPreal™ qPCR 2 X PreMIX (SYBR Green with high ROX). The mixture was transferred to Zipperstrip Strip PCR Tubes and subjected to DNA amplification in a thermal cycler (Bio-Rad). After completion of the amplification, the mixture was transferred to a microcentrifuge tube and 2 μl of the sample, 5 μl of 2 X mastermix, 2.5 μl of pure water, and 0.5 μl of primers were added to the PCR plate. Then, mGAPDH and mMSTN were each added to one sample. The resulting mixture was sealed and subjected to real-time PCR (Applied Biosystems, Quantstudio3) to detect amplified DNAs.

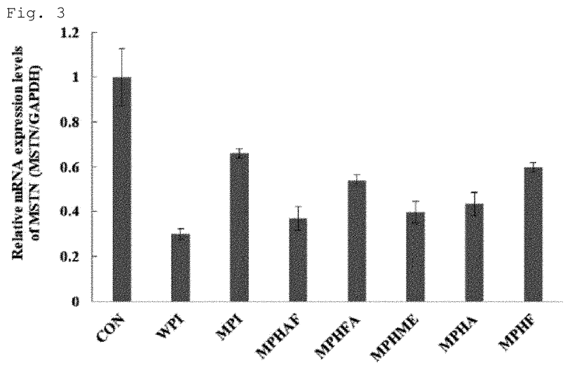

As can be seen in FIG. 3 and Table 3, mealworm protein hydrolysates (MPHs) expressed myostatin at lower levels by hydrolysis condition than the mealworm protein isolate (MPI), demonstrating their alleviative effect on sarcopenia. Among them, MPHAF exhibited the lowest expression level of myostatin.

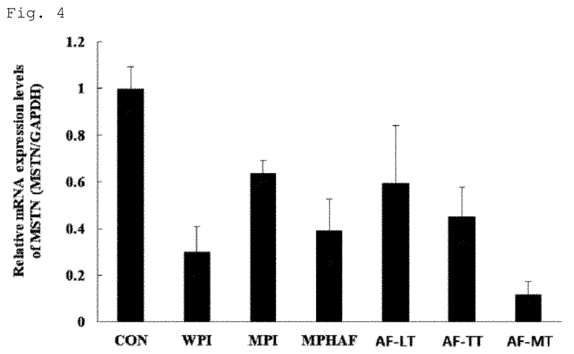

Size fractions of the MPHAF which exhibited the lowest myostatin expression level were examined for myostatin expression level. As seen in FIG. 4 and Table 4, the myostatin expression level in the hydrolysate with a molecular weight of 10 kDa or greater (AF-MT) was the lowest among those in the fractions and was identified to be lower than that in WPI, indicating the highest effect of alleviating sarcopenia.

TABLE 3
Relative Myostatin Expression Level (MSTN/GAPDH)
After Treatment of Myoblast (C2C12) with Mealworm
Protein Hydrolysate According to Hydrolysis Condition

| Sample | Mean | Standard deviation |
|---|---|---|
| CON | 1 | 0.223 |
| WPI | 0.299 | 0.04 |
| MPI | 0.661 | 0.036 |
| MPHAF | 0.369 | 0.093 |
| MPHFA | 0.54 | 0.041 |
| MPHME | 0.397 | 0.082 |
| MPHA | 0.434 | 0.091 |
| MPHF | 0.597 | 0.04 |

TABLE 4
Relative Myostatin Expression Level (MSTN/GAPDH)
After Treatment of Myoblast (C2C12) with
MPHAF According to Molecular Size

| | | Standard |
|---|---|---|
| Sample | Mean | deviation |
| CON | 1 | 0.16 |
| WPI | 0.299 | 0.189 |
| MPI | 0.638 | 0.094 |
| MPHAF | 0.389 | 0.238 |
| AF-LT | 0.594 | 0.429 |
| AF-TT | 0.452 | 0.219 |
| AF-MT | 0.118 | 0.096 |

<1-3>Relative Activity of Myostatin Promoter

Effects of MPI and MPH on the activity of a myostatin promoter were measured using a gene assay based on the dual luminescence of luciferase and beta-galactosidase. In this regard, luciferase activity assay was conducted prior to beta-galatosidase quantitative analysis to increase the accuracy of gene expression analysis.

In brief, a MSTN (myostatin) promoter (−534˜+132, 667 bp) was inserted into pGL4.15 vector using Xhol and BglII. Cells were seeded at a density of 4×105 cells/well into 12-well plates. After 24 hours, the cells were washed with PBS and 900 μl of Opti-MEM medium was added to each well. pGL4.15-MSTN promoter (1 μg, firfly luc) and pRL-TK (200 ng, Renilla luc) were mixed together with Lipofectamine 2000 (2 μl, Invitrogen) in 100 μl of Opti-MEM mediuma and, for co-transfection, the mixture was added in an amount of 100 μl to the cells in each well. In this regard, a pGL4.15 empty vector was used as a negative control. Four hours later, the medium was replaced by a fresh one. After 12 hours, luciferase activity (luminescence) was measured using Dual-Luciferase® Reporter Assay System (Promega) and luminometer (EnSpire Mpltimode Plate Reader, PerkinElmer). pRL-TK was used as a loading control.

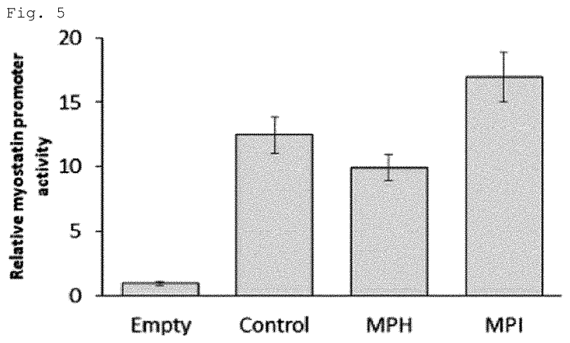

The result is depicted in FIG. 5. As can be seen, MPI exhibited higher myostatin promoter activity than the control while MPH was lower in myostatin promoter activity than the control. Thus, MPH is considered to have a higher effect of alleviating sarcopenia than MPI by decreasing the activity of myostatin, which inhibits muscle synthesis.

For reference, the MPH in FIG. 5 was obtained by treating the substrate with 1% alcalase for 12 hours (the same conditions as for MPHA, except for treatment time).

### Experimental Example 2

Assay for Inhibitory Activity against Inflammatory Cell Expression (Anti-Inflammatory Effect)

To examine inhibitory rates against inflammatory cell expression of MPI and MPH samples according to hydrolysis condition and size, macrophages were observed for expression levels of IL-6, TNF-α, and IL-1b, which are blood inflammation-causing cytokines, after treatment with LPS. As a positive control, a whey protein isolate (WPI) was used while a mixture of lipopolysaccharide (LPS) and distilled water used as a negative control. In other words, inflammation was induced by LPS and expression levels of the inflammatory cytokines IL-6, TNF-α, and IL-1b were observed in the presence of the samples at various concentrations. For reference, blood inflammation-causing cytokines promote the degradation of myofibrillar protein to lower protein synthesis, resulting in direct muscle consumption.

Prior to measurement of expression levels of inflammatory cytokines, protein levels in each sample were measured by the Bradford assay. Briefly, 10-fold serial dilutions of the sample were each mixed in an amount of 100 μl with 5 ml of the assay reagent, followed by reading absorbance at 595 nm. A calibration curve was constructed by reading absorbance of mixtures in which a 1 mg/mL γ-globulin standard solution was diluted into concentrations of 10-100 μl/100 μl and mixed with the reagent in the same manner as in the sample.

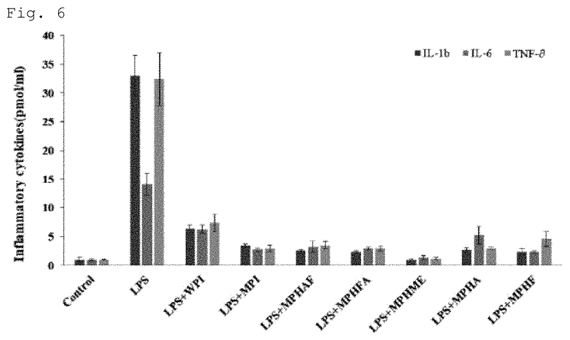

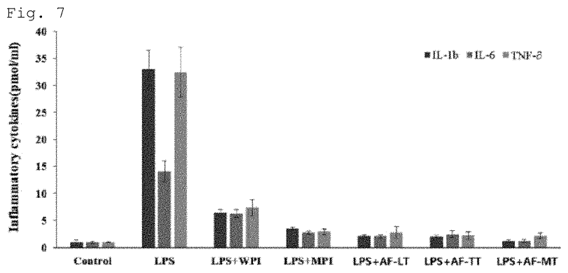

As shown in FIGS. 6 and 7, when treated with the inflammation factor LPS, macrophages expressed elevated levels of the inflammatory cytokines IL-6, TNF-α, and IL-1b. In all test groups treated with the samples together with LPS, the expression levels of the inflammatory cytokines were decreased in a dose-dependent manner. From the data, it was understood that all the samples reduced the expression of inflammatory cytokines. In detail, the samples exhibited higher inhibitory activity against IL-1b in all test groups, compared to the positive control

(WPI). Particularly, the highest inhibitory activity against TNF-α expression was observed in the test group treated with MPHME, compared to the positive control (WPI). Higher inhibitory effects on IL-6 were measured in all the test groups, except for the MPHA- or MPHAF-treated group, than the positive control (WPI).

From the data obtained above, it was understood that the hydrolysate MPHME highly inhibited expression of all of TNF-α, IL-1b, and IL-6, showing the highest anti-inflammatory effect.

### Experimental Example 3

Hydrolysis Degree, Amino Acid Composition, and Molecular Weight of Mealworm Protein Hydrolysate

<3-1>TNBS Assay

In this experiment, degrees of hydrolysis of the hydrolysates prepared under different hydrolysis conditions (see Example <2-5>) were measured. To this end, the degree of hydrolysis of each protein hydrolysate was determined by measuring concentrations of available amino groups which are used as an index therefor. For reference, when a protein is hydrolyzed, available amino groups increase in proportion to the degree of hydrolysis and thus can be used as an index indicative of a content of peptides.

Briefly, degrees of hydrolysis of the MPH prepared according to conditions were measured using TNBS (2,4,6-trinitrobenzene sulfonic acid) solution. Standard solutions with concentrations of 2-20 μg/ml were prepared using 0.1M sodium bicarbonate (pH 8.5) solution (reaction buffer, RB) and L-leucine. Then, TNBS solution (working reagent, WR) with a concentration of 0.01% (v/v) was prepared using RB. To 500 μl of each of the sample and the standard solution was added 250 μl of WR, followed by incubation at 37° C. for 2 hours. Then, the reaction was stopped with 250 μl of 10% SDS and 125 μl of 1N HCl and absorbance was measured at 335 nm.

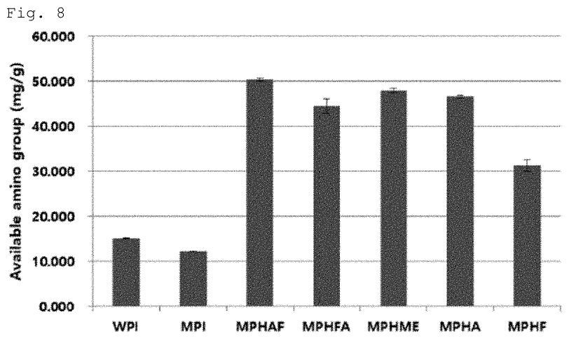

Among hydrolysates by hydrolysis conditions, As shown in FIG. 8 and Table 5, MPHF (1%, flavourzyme, 24 h) showed the lowest available amino group concentration (31.283 mg/g). On the other hand, MPHAF ((0.5%, alcalase, 12 h)→(0.5%, flavorzyme, 12 h)) showed the highest available amino group concentration (50.323 mg/g). Thus, it was confirmed that the enzyme treatment under the MPHAF condition hydrolyzes mealworms at the highest degree.

TABLE 5
Concentration of Available Amino Group
(mg/g) by Hydrolysis Condition

| | Conc. of | |
|---|---|---|
| | available amino | Standard |
| Sample | group (mg/g) | deviation |
| WPI | 15.008 | 0.139 |
| MPI | 12.139 | 0.276 |
| MPHAF | 50.323 | 0.643 |
| MPHFA | 44.412 | 2.771 |
| MPHME | 47.912 | 0.96 |
| MPHA | 46.556 | 0.547 |
| MPHF | 31.283 | 2.355 |

<3-2>BCA Assay

To measure degrees of hydrolysis of the hydrolysates prepared under different hydrolysis conditions (see Example <2-5>), the protein was monitored for structural change by BCA assay.

In brief, the BCA assay reagents A and B were mixed at a ratio of 50:1 to prepare a working reagent (WR) and BSA standard solutions with different concentrations (0-2000 μg/ml). WR was added in an amount of 2 ml to 0.1 ml of each of the standard solutions and the sample, followed by incubation at 37° C. for 30 minutes. After incubation, the mixture was cooled for 10 minutes and measured for absorbance at 562 nm.

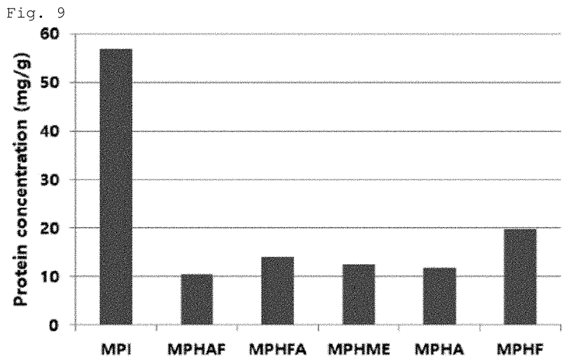

As shown in FIG. 9 and Table 6, MPI (56.882 mg/g) had higher protein content than MPH (MPHF: 19.84 mg/g, MPHFA: 14.02 mg/g, MPHME: 12.43 mg/g, MPHA: 11.88 mg/g, MPHAF: 10.52 mg/g) and MPHAF showed the lowest protein concentration among MPH, indicating that the hydrolysis conditions of MPHAF was the most effective for hydrolysis.

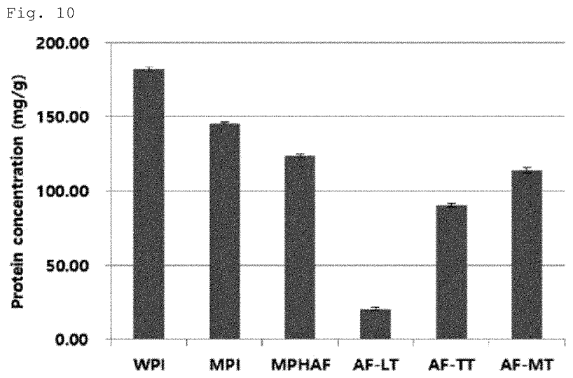

Afterwards, the protein content of fractions obtained by fractionating MPHAF with the highest degree of hydrolysis by molecular size was measured. As shown in FIG. 10 and Table 7, WPI (182.19 mg/g) was the highest in protein content, followed by MPI (145.69 mg/g), MPHAF (123.66 mg/g), and the fractions (AF-MT:113.97 mg/g, AF-TT:90.19 mg/g, AF-LT:20.45 mg/g) in the order.

TABLE 6
Protein Concentration of Mealworm Protein
Hydrolysate by Hydrolysis Condition (mg/g)

| Sample | BCA Conc. (mg/g) |
|---|---|
| MPI | 56.882 |
| MPHAF | 10.518 |
| MPHFA | 14.018 |
| MPHME | 12.427 |
| MPHA | 11.882 |
| MPHF | 19.836 |

TABLE 7
Protein Concentration of Hydrolysate Fraction
of MPHAF by Molecular Size (mg/g)

| | | Standard |
|---|---|---|
| Sample | Mean | deviation |
| WPI | 182.19 | 2.107 |
| MPI | 145.69 | 1.157 |
| MPHAF | 123.66 | 2.258 |
| AF-LT | 20.45 | 1.637 |
| AF-TT | 90.19 | 2.332 |
| AF-MT | 113.97 | 3.388 |

<3-3>Analysis of Constituent Amino Acid

In this experiment, inhibitory effects of constituent amino acids on sarcopenia were examined. In this regard, fractions fractionated according to molecular size from MPHAF, which exhibited the highest degree of hydrolysis in the enzymatic hydrolysis, were analyzed for amino acid composition and content by HPLC.

First, 0.05 g of the sample was placed in a digestion tube and subjected to acid hydrolysis at 105° C. for 24 hours in the presence of 2 ml of 6N HCl under a vacuum condition. Thereafter, the mixture was diluted 100 times in a distilled water and filtered through a 0.45-nm nylon syringe filter. The filtrate was used as an analyte. Conditions of amino acid analysis instrument were as listed in Table 8, below. After unit correction for the experimental results, the contents of 17 amino acids were measured, and the results are shown in detail in Table 9.

TABLE 8
Condition of Amino Acid Analyzer

| Measurement | Condition |
|---|---|
| Instrument | YL9100 HPLC System, Young in Chromass |
| | Co. Anyang, Korea |
| Column | HPLC Column, 3.9 mm × 150 mm |
| Column temp. | 37° C. |
| Mobile phase | A: 10% Warters accq-tag Eluent A concentrate |
| | B: 60% ACN |
| Wave length | Ex: 250 mm, Em: 395 mm |

TABLE 9
Constituent Amino Acid of Hydrolysate Fractionated from MPHAF by Molecular Size

| | WPI | | MPI | | AF-LT | | AF-TT | | AF-MT | |
|---|---|---|---|---|---|---|---|---|---|---|
| Amino acid | Mean | SD | Mean | SD | Mean | SD | Mean | SD | Mean | SD |
| Histidine | 1.693 | 0.682 | 2.385 | 0.605 | 0.754 | 0.1 | 1.003 | 0.179 | 1.234 | 0.048 |
| Isoleucine | 3.908 | 0.364 | 4.086 | 0.112 | 1.343 | 0.033 | 1.852 | 0.052 | 1.871 | 0.062 |
| Leucine | 9.125 | 0.31 | 8.35 | 0.178 | 3 | 0.08 | 3.841 | 0.053 | 3.832 | 0.076 |
| Lysine | 8.04 | 1.093 | 5.505 | 0.343 | 2.471 | 0.045 | 3.004 | 0.175 | 2.952 | 0.059 |
| Methionine | 2.57 | 0.087 | 0.787 | 0.045 | 1.253 | 0.024 | 0.804 | 0.016 | 0.712 | 0.07 |
| Phenylalanine | 2.455 | 0.065 | 4.136 | 0.177 | 1.354 | 0.026 | 1.886 | 0.061 | 1.937 | 0.08 |
| Tryptophan | ND | ND | ND | ND | ND | ND | ND | ND | ND | ND |
| Threonine | 6.528 | 0.546 | 3.653 | 0.185 | 1.02 | 0.067 | 1.483 | 0.05 | 1.555 | 0.105 |
| Valine | 3.843 | 0.087 | 3.645 | 0.113 | 1.047 | 0.02 | 2.292 | 0.041 | 2.285 | 0.048 |
| Sum of | 38.161 | 1.812 | 32.547 | 0.773 | 12.243 | 0.234 | 16.166 | 0.142 | 16.378 | 0.345 |
| essential | | | | | | | | | | |
| amino acid | | | | | | | | | | |
| Alanine | 4.504 | 0.059 | 7.194 | 0.278 | 3.012 | 0.153 | 3.48 | 0.153 | 3.362 | 0.102 |
| Arginine | 1.26 | 0.096 | 4.63 | 0.608 | 2.226 | 0.135 | 2.288 | 0.074 | 2.334 | 0.082 |
| Aspartic acid | 10.452 | 0.805 | 10.786 | 0.087 | 1.523 | 0.169 | 4.649 | 0.233 | 4.847 | 0.387 |
| Cysteine | 0.177 | 0.001 | 0.607 | 0.032 | 0.255 | 0.007 | 0.311 | 0.004 | 0.299 | 0.009 |
| Glutamic acid | 16.425 | 1.371 | 12.459 | 0.839 | 2.743 | 0.052 | 5.827 | 0.107 | 5.889 | 0.25 |
| Glycine | 1.673 | 0.353 | 7.559 | 0.544 | 2.715 | 0.025 | 3.348 | 0.018 | 3.324 | 0.113 |
| Proline | 5.886 | 0.153 | 5.949 | 0.242 | 1.592 | 0.046 | 2.478 | 0.076 | 2.456 | 0.064 |
| Serine | 5.483 | 0.369 | 6.059 | 0.246 | 1.644 | 0.022 | 2.648 | 0.034 | 2.587 | 0.102 |
| Tyrosine | 2.493 | 0.009 | 8.536 | 0.445 | 3.59 | 0.105 | 4.375 | 0.062 | 4.208 | 0.121 |
| Total of | 86.514 | 3.864 | 96.325 | 1.81 | 31.543 | 3.88 | 45.57 | 2.014 | 45.684 | 2.252 |
| Amino Acid | | | | | | | | | | |
| Hydrophobic | 33.963 | 1.275 | 41.706 | 2.263 | 15.316 | 0.539 | 19.981 | 0.552 | 19.779 | 0.586 |
| amino acid | | | | | | | | | | |
| Hydrophilic | 41.558 | 3.084 | 42.1 | 1.019 | 10.775 | 0.349 | 19.294 | 0.275 | 19.385 | 0.843 |
| amino acid | | | | | | | | | | |
| [symbol] | | | | | | | | | | |
| BCAA | 16.875 | 0.758 | 16.081 | 0.245 | 5.391 | 0.133 | 7.986 | 0.133 | 7.989 | 0.192 |

<3-4>Sds-Page

Low-molecular weight peptides produced by degeneration of the hydrolysates prepared under different hydrolysis conditions (see Example <2-5>) were quantitatively analyzed by SDS-PAGE.

In brief, with reference to the BCA assay results, 0.057 g of each sample was added to 5 ml of distilled water to afford a protein content of 50 μg/10 p1. After 10 p1 of the sample was mixed with 10 p1 of 2× laemmli buffer, 5p1 of mercaptoethanol was added thereto. The mixture was boiled at 95° C. for 5 minutes. The pretreated sample was loaded into a 15% SDS gel and electrophoresed at 80V-120V. After completion of the electrophoresis, the gel was stained with Coomassie brilliant blue buffer and destained with Coomassie brilliant blue R-250 destaining buffer containing acetic acid and methanol. Subsequently, the polyacrylamide gel was scanned to obtain scanning images.

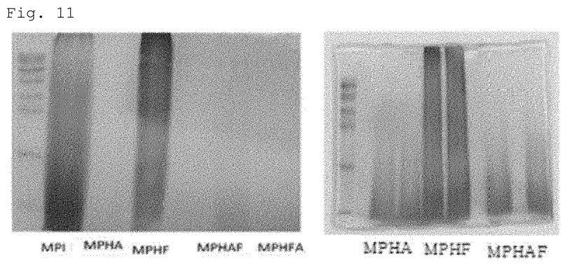

As can be understood from the patterns in FIG. 11, among the hydrolysate, MPHF contained high molecular weight peptides whereas peptides with a molecular weight of 6 kDa or less were most abundant in MPHAF.

### Experimental Example 4

Assay for Antioxidative Activity

In this experiment, effects of the hydrolysates produced by enzymatic treatments on oxidative stress, which may affect sarcopenia, were measured using an ABTS radical scavenging assay.

Briefly, 7.4 mM ABTS and potassium persulfate were mixed at a ratio of 1:1 and stored in a cooled and dark place for 24 hours to form radicals in the reagent. Subsequently, ABTS reagent was diluted with distilled water so that absorbance of 0.7±0.02 was measured at 734 nm wavelength using a spectrophotometer. Each sample was diluted in distilled water to make a concentration of 500 μg/ml. The dilution was mixed at a ratio of 1:1 with ABTS reagent and stored for 10 minutes in a cooled and dark place before reading absorbance at 760 nm. In this regard, the sample extraction solution and distilled water were mixed at a ratio of 1:1 for an empty sample while a control was prepared by mixing the ABTS reagent and distilled water at a ratio of 1:1. ABTS radical scavenging rates (%) were calculated using the read absorbance.

$$Scavenging rate ( \% ) = ( 1 - \frac{A b s o r bance of sample - a b s o r bance of empty sample}{a b s o r bance of control} \times 100$$

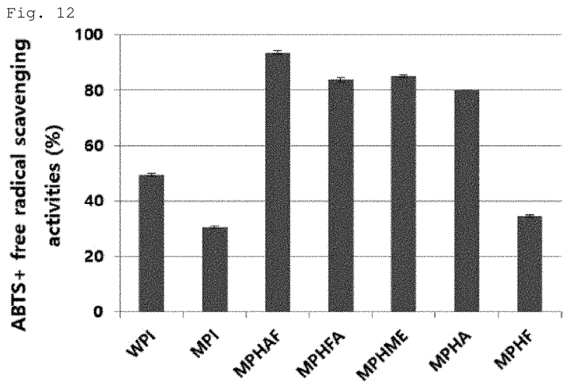

As shown in FIG. 12 and Table 10, MPH obtained according hydrolysis condition showed high ABTS radical scavenging ability, compared with MPI, demonstrating its high antioxidant activity. Of the hydrolysates, MPHAF was measured to have the highest antioxidative activity (93.55%).

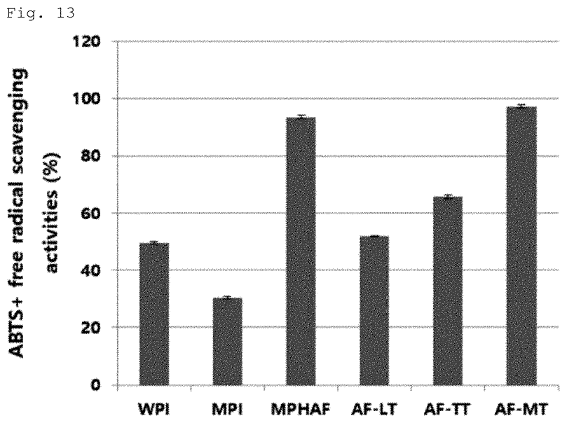

Thereafter, the ABTS radical scavenging ability of the fractions fractionated by molecular size from MPHAF was measured and the results are depicted in FIG. 13 and summarized in Table 11. As can be seen, it was confirmed that the fraction (AF-MT 97.24%) of 10 kDa or more had the highest antioxidant activity.

TABLE 10
ABTS Radical Scavenging Rate (%) of Mealworm
Protein Hydrolysate According to Hydrolysis Condition

| | | Standard |
|---|---|---|
| Sample | Mean | deviation |
| WPI | 49.508 | 0.835 |
| MPI | 30.4 | 0.783 |
| MPHAF | 93.546 | 1.132 |
| MPHFA | 83.73 | 1.513 |
| MPHME | 84.983 | 0.673 |
| MPHA | 80.162 | 0.095 |
| MPHF | 34.443 | 0.72 |

TABLE 11
ABTS Radical Scavenging Rate (%) of
Size-Fractioned Hydrolysate of MPHAF

| | | Standard |
|---|---|---|
| Sample | Mean | deviation |
| WPI | 49.508 | 0.835 |
| MPI | 30.4 | 0.783 |
| MPHAF | 93.546 | 1.132 |
| AF-LT | 51.887 | 0.287 |
| AF-TT | 65.779 | 1.282 |
| AF-MT | 97.241 | 0.989 |

The preferred embodiments of the disclosure have been explained so far. a person skilled in the art will understand that the invention may be implemented in modifications without departing from the basic characteristics of the disclosure. Accordingly, the foregoing exemplary embodiments and advantages are merely exemplary and are not to be construed as limiting the present disclosure. The present teaching can be readily applied to other types of apparatuses. Also, the description of the exemplary embodiments of the present inventive concept is intended to be illustrative, and not to limit the scope of the claims.

### DESCRIPTION OF ABBREVIATIONS

WPI: Whey protein isolate

MPI: Mealworm Protein Isolate

MPHA: Hydrolysate obtained by treatment with 1% alcalase for 24 hours

MPHF: Hydrolysate obtained by treatment with 1% flavourzyme for 24 hours

MPHME: Hydrolysate obtained by treatment with 1% combined enzymes (0.5% (w/v) alcalase & 0.5% (w/v) flavourzyme) for 24 hours

MPHAF: Hydrolysate obtained by treatment with 0.5% (w/v) alcalase for 12 hours and then with 0.5% (w/v) flavourzyme for 12 hours

MPHFA: Hydrolysate obtained by treatment with 0.5% (w/v) flavourzyme for 12 hours and then with 0.5% (w/v) alcalase for 12 hours

AF-LT: Hydrolysate with molecular weight of 3 kDa or less in MPHAF

AF-TT: Hydrolysate with molecular weight of 3-10 KDa in MPHAF

AF-MT: Hydrolysate with molecular weight of 10 KDa or higher in MPHAF

1. A food composition comprising a mealworm protein isolate or a hydrolysate thereof as an active ingredient for prevention or alleviation of a muscular disease.

2. The food composition of claim 1, wherein the mealworm protein isolate is prepared by a process comprising the steps of: a) pulverizing a dried mealworm larva mass; b) defat the pulverized mass by addition of ethanol thereto; c) adding and mixing the defatted mealworm larva mass with sodium hydroxide, followed by centrifugation to obtain a pellet; and d) desalting the pellet, followed by lyophilization.

3. The food composition of claim 1, wherein the hydrolysate is prepared by treating the mealworm protein isolate with an alcalase, flavourzyme, or a combination thereof.

4. The food composition of claim 1, wherein the mealworm protein isolate or the hydrolysate thereof inhibits expression of myostatin.

5. The food composition of claim 1, wherein the muscular disease is a muscular disease caused by muscle dysfunction, muscle loss, muscle atrophy, muscle wasting, or muscle degeneration.

6. The food composition of claim 5, wherein the muscular disease is selected from the group consisting of atony, muscular atrophy, muscular dystrophy, myasthenia, cachexia, rigid spine syndrome, amyotrophic lateral sclerosis (Lou Gehrig's disease), Charcot-Marie-Tooth disease, sarcopenia, and a combination thereof.

7. A health functional food comprising a mealworm protein isolate or a hydrolysate thereof as an active ingredient for prevention or alleviation of a muscular disease.

8. The health functional food of claim 7, wherein the health functional food is selected from the group consisting of beverages, meats, confectionaries, noodles, rice cakes, breads, gums, candies, ice creams, and liquors.

9. A pharmaceutical composition comprising a mealworm protein isolate or a hydrolysate thereof as an active ingredient for prevention or treatment of a muscular disease.

10. (canceled)

11. The food composition of claim 2, wherein the muscular disease is a muscular disease caused by muscle dysfunction, muscle loss, muscle atrophy, muscle wasting, or muscle degeneration.

12. The food composition of claim 3, wherein the muscular disease is a muscular disease caused by muscle dysfunction, muscle loss, muscle atrophy, muscle wasting, or muscle degeneration.

13. The food composition of claim 4, wherein the muscular disease is a muscular disease caused by muscle dysfunction, muscle loss, muscle atrophy, muscle wasting, or muscle degeneration.
